(19) United States
(12) Patent Application Publication
(10) Pub. No.: US 2009/0011007 A1
(43) Pub. Date: Jan. 8, 2009

(54) Pharmaceutical Compositions Containing Mixtures of Polymers and Active Agents Poorly Soluble in Water

(75) Inventors: Christian Meier, Darmstadt (DE); Kathrin Nollenberger, Frankfurt (DE); Andreas Gryczke, Riedstadt (DE); Hans-Ulrich Petereit, Darmstadt (DE); Jennifer Dressman, Frankfurt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Evonik Roehm GmbH, Darmstadt (DE)

(21) Appl. No.: 12/159,538

(22) PCT Filed: Jan. 17, 2007
(86) PCT No.: PCT/EP2007/050465

§ 371 Date: Jun. 27, 2008

(30) Foreign Application Priority Data
Feb. 3, 2006 (DE) 10 2006 005 485.7

(51) Int. Cl. A61K 9/48; A61K 47/00; A61K 47/38; A61K 31/4418; A61K 9/14; A61K 9/20

(52) U.S. Cl. 424/451; 514/772.3; 514/781; 424/464; 424/489; 514/356

(57) ABSTRACT

The invention relates to a pharmaceutical composition comprising a mixture of at least one cationic, water-soluble (meth)acrylate copolymer, at least one water-insoluble polymer and at least one active ingredient having a solubility in demineralized water of 3.3 g/l or less, characterized in that the water-insoluble polymer and the active ingredient are present in a ratio of at most 3.5 to 1 parts by weight, and the pharmaceutical composition has the property of releasing the active ingredient present in a medium buffered to pH 1.2 in dissolved form in a concentration which, after 2 hours at pH 1.2, corresponds to at least sixteen times the solubility value of the active ingredient alone at pH 1.2.

The invention relates to various pharmaceutical compositions comprising mixtures of polymers and active ingredients sparingly soluble in water.

### STATE OF THE ART

EP 0 058 765 B1 describes swellable coating compositions soluble in gastric juice and their use in a process for coating pharmaceutical forms. They are in particular water-soluble (meth)acrylate copolymers which are composed partly or fully of alkyl acrylates and/or alkyl methacrylates having a tertiary amino group in the alkyl radical.

U.S. Pat. No. 6,391,338 describes the improvement in solubility or increase in bioavailability of essentially water-insoluble active ingredients, for example ibuprofen, itraconazole and nifedipine by means of flash-flow or extrusion of the active ingredients and polymers of the EUDRAGIT® E type. During the processing, the active ingredients can be converted to an energetically higher state (solid dispersion) and then released in the form of nanoparticles in a dissolved state.

U.S. Pat. No. 6,319,520 describes pharmaceutical compositions for controlled active ingredient release, consisting of thermally shapable mixtures of at least one active ingredient and one or more pH-independent polymers from the group of the polymethacrylates. The pharmaceutical compositions can be prepared by means of injection moulding, injection co-moulding, extrusion or coextrusion. Preferred (meth)acrylate copolymers are EUDRAGIT® RL and RS, which may optionally also be used together with EUDRAGIT® E or EUDRAGIT® L100, L100-55 and/or S100. In the examples, the active ingredients including benfluorex hydrochloride, rilmetidine dihydrogen, fenspirid hydrochloride are processed with EUDRAGIT® RL, RS and mixtures thereof by means of extrusion or injection moulding.

WO 01/39751 A1 describes a process for producing mouldings by means of injection moulding. The process steps comprise
a) melting of a (meth)acrylate copolymer which is composed of 30 to 80% by weight of free-radically polymerized C1- to C4-alkyl esters of acrylic acid or of methacrylic acid and 70 to 20% by weight of (meth)acrylate monomers having a tertiary ammonium group in the alkyl radical, the (meth)acrylate copolymer being present in a mixture with 1 to 70% by weight of a plasticizer and a drier in a ratio of 1:1 to 1:20, at least 1% by weight of plasticizer being present, and 0.05 to 5% by weight of a release agent are also present and additionally further customary additives or excipients and optionally an active pharmaceutical ingredient may be present in the mixture, and the mixture, before the melting, has a content of low-boiling constituents having a vapour pressure of at least 1.9 bar at 120° C. of over 0.5% by weight,
b) degassing the mixture in the thermoplastic state at temperatures of at least 120° C., which lowers the content of the low-boiling constituents having a vapour pressure of at least 1.9 bar at 120° C. to at most 0.5% by weight and
c) injecting the molten and degassed mixture into the shaping cavity of an injection mould, the shaping cavity having a temperature which is at least 10° C. below the glass transition temperature of the (meth)acrylate copolymer, cooling the melt mixture and removing the resulting moulding from the mould.

The (meth)acrylate copolymer, which may preferably be an EUDRAGIT® E, may be present in a mixture with further polymers to control the active ingredient release. The content of further polymers should not be more than 20% by weight, preferably at most 10% by weight, in particular 0-5% by weight. Further polymers for mixtures include EUDRAGIT® NE 30 D, EUDRAGIT® RS and EUDRAGIT® RL. The process can be applied to any active ingredients, and ranitidine is one mentioned.

WO 01/43935 A2 describes a process for producing mouldings by means of injection moulding, comprising the process steps of

A) melting a mixture of
a) a (meth)acrylate copolymer which is composed of 40 to 100% by weight of free-radically polymerized C₁- to C₄-alkyl esters of acrylic acid or of methacrylic acid and 0 to 60% by weight of (meth)acrylate monomers having an anionic group in the alkyl radical, which contains
b) 0.1 to 3% by weight of a release agent, and optionally
c) 0 to 50% by weight of a drier
d) 0 to 30% by weight of a plasticizer
e) 0 to 100% by weight of additives or excipients
f) 0 to 100% by weight of an active pharmaceutical ingredient
g) 0 to 20% by weight of a further polymer or copolymer
   may be present in the mixture, the amounts of components b) to g) being based on the (meth)acrylate copolymer a) and the mixture, before the melting, having a content of low-boiling constituents having a vapour pressure of at least 1.9 bar at 120° C. of over 0.5% by weight,
   B) degassing the mixture in the thermoplastic state at temperatures of at least 120° C., which lowers the content of the low-boiling constituents having a vapour pressure of at least 1.9 bar at 120° C. to at most 0.5% by weight,
   C) injecting the molten and degassed mixture into the shaping cavity of an injection mould, the shaping cavity having a temperature which is at least 10° C. below the glass transition temperature of the (meth)acrylate copolymer, cooling the melt mixture and removing the resulting moulding from the mould.

The mixture may contain 0 to 20% by weight of a further polymer or copolymer g). To control the active ingredient release, it may be advantageous in the individual case to add further polymers. The content of further polymers in the mixture is, however, not more than 20% by weight, preferably at most 10% by weight, in particular 0-5% by weight, based on the (meth)acrylate copolymer.

Examples of such further polymers are: polyvinylpyrrolidones, polyvinyl alcohols, cationic (meth)acrylate copolymers of methyl methacrylate and/or ethyl acrylate and 2-dimethylaminoethyl methacrylate (EUDRAGIT® E100), carboxymethylcellulose salts, hydroxypropylcellulose (HPMC), neutral (meth)acrylate copolymers of methyl methacrylate and ethyl acrylate (dry substance formed from EUDRAGIT® NE 30 D), copolymers of methyl methacrylate and butyl methacrylate (PLASTOID® B) or (meth)acrylate copolymers having quaternary ammonium groups, containing trimethylammonioethyl methacrylate chloride as a monomer (EUDRAGIT® RL and EUDRAGIT® RS).

WO 2004/019918 describes a process for preparing a granule or powder suitable as a coating composition and binder for oral or dermal pharmaceutical forms, for cosmetics or dietary supplements, consisting essentially of (a) a copolymer consisting of free-radically polymerized C1- to C4-esters of acrylic acid or methacrylic acid and further (meth)acrylate monomers which have functional tertiary amino groups, (b) 3 to 25% by weight, based on (a), of an emulsifier having an HLB value of at least 14, (c) 5 to 50% by weight, based on (a), of a C₁₂- to C₁₈-monocarboxylic acid or of a C₁₂- to C₁₈-hydroxyl compound, components (a), (b) and (c) being combined or mixed with one another simultaneously or successively optionally with addition of an active pharmaceutical ingredient and/or further customary additives, melted in a heatable mixer and mixed, and the melt is cooled and comminuted to a granule or powder. The granules and powders obtained by the process are suitable in particular for the formulation of moisture-sensitive active pharmaceutical ingredients, for example acetylsalicylic acid, carbenoxolone, cefalotin, epinephrine, imipramine, potassium iodide, ketoprofen, levodopa, nitrazepam, nitroprusside, oxitetracyclin-HCl, promethazine, omeprazole or other benzimidazole derivatives, ranitidine or streptomycin.

### PROBLEM AND SOLUTION

U.S. Pat. No. 6,391,338 describes the improvement in solubility or increase in bioavailability of essentially water-insoluble active ingredients, for example ibuprofen, itraconazole and nifedipine by means of flash-flow or extrusion of the active ingredients and polymers of the EUDRAGIT® E type. During the processing, the active ingredients can be converted to an energetically higher state (solid dispersion) and then released in the form of nanoparticles in a dissolved state. This is a scientifically remarkable approach which leads to good results in many cases.

Pharmaceutical compositions formulated according to U.S. Pat. No. 6,391,338 generally have the property of releasing an active ingredient present which has a solubility in demineralized water of 3.3 g/l or less, after dissolution of an EUDRAGIT® E matrix at acidic pH, in dissolved form in a concentration which initially corresponds to at least twice the solubility value of the active ingredient in demineralized water.

However, the inventors have found that this effect continues only over a relatively short period. After the initial rise in concentration of the measurably dissolved active ingredient, it falls again below the limit of twice the solubility value of the active ingredient in demineralized water. The measurement of the dissolved active ingredient can be monitored, depending on the active ingredient type or active ingredient nature, for example, by means of chromatographic or spectroscopic methods, for example UV measurement or HPLC, or by other methods. The inventors suspect that the initially higher energetic state of the active ingredient, after the dissolution of the EUDRAGIT® E matrix, degenerates rapidly again and the active ingredient is converted to a sparingly soluble or insoluble form which is then at best bioavailable to a limited degree, if at all, and possibly even crystallizes, aggregates and/or precipitates. This proportion of the active ingredient is therefore available only to a limited degree when it is transferred into the duodenum. There is the risk that the originally desired blood levels are not attained.

In the case of active ingredients sparingly soluble in water, which are intended to be released and absorbed again immediately in the stomach or after passing through the stomach, for which a certain blood level has to be attained for therapeutic action, the problem thus exists that this blood level often cannot be attained, since the active ingredient recrystallizes or precipitates again too rapidly, and its originally increased bioavailability is thus lost again.

WO 01/39751 A1 describes a process for producing mouldings by means of injection moulding. In particular, the object of providing (meth)acrylate copolymers with tertiary amino groups in a form processible in injection moulding should be achieved, such that corresponding mouldings are obtained in pharmaceutical quality. It is mentioned that, as well as (meth)acrylate copolymers with tertiary amino groups alone, it is also possible to process mixtures with EUDRAGIT® NE, EUDRAGIT® RS or RL. Examples of such mixtures alone or in combination with active ingredients are not present. WO 01/39751 A1 does not provide a person skilled in the art with any indications to the solution of the abovementioned problem, that of bringing about a relatively long-lasting improvement in solubility for active ingredients sparingly soluble in water.

WO 01/43935 A2 describes a process for producing mouldings by means of injection moulding. In particular, the object of providing (meth)acrylate copolymers with anionic groups in a form processible in injection moulding should be achieved, such that corresponding mouldings obtained in pharmaceutical quality. It is mentioned that, as well as (meth)acrylate copolymers with anionic groups alone, it is also possible to process mixtures with EUDRAGIT® NE, EUDRAGIT® RS or RL. Examples of such mixtures alone or in combination with active ingredients are not present. WO 01/43935 A2 does not provide a person skilled in the art with any indications to the solution of the abovementioned problem, that of bringing about a relatively long-lasting improvement in solubility for active ingredients sparingly soluble in water.

Proceeding from the prior art, the intention is therefore to provide a pharmaceutical formulation for active ingredients sparingly soluble in water, for which an enhanced solubility and associated bioavailability of the active ingredient in a gastric juice-like environment, pH 1.2, is attained and remains entirely or at least partly stable over a period of at least 120 minutes. The gastric juice-like test environment represents the high requirement, so that it can be assumed that the state of elevated solubility, when it can be attained in a stable manner in vitro at pH 1.2 after 120 min, no longer changes significantly in a disadvantageous manner even in vivo after transfer into the section of the intestine at the higher pH values which exist there.

The object is achieved by a

pharmaceutical composition comprising a mixture of at least one cationic, water-soluble (meth)acrylate copolymer, at least one water-insoluble polymer and at least one active ingredient having a solubility in demineralized water of 3.3 g/l or less,
characterized in that
   the water-insoluble polymer and the active ingredient are present in a ratio of at most 3.5 to 1 parts by weight, and the pharmaceutical composition has the property of releasing the active ingredient present in a medium buffered to pH 1.2 in dissolved form in a concentration which, after 2 hours at pH 1.2, corresponds to at least sixteen times the solubility value of the active ingredient alone at pH 1.2.

The invention further relates to two alternative processes for preparing the inventive pharmaceutical compositions.

The invention further relates to a process for producing a pharmaceutical form comprising the inventive pharmaceutical composition, and to the resulting pharmaceutical form.

The invention further relates to the use of the inventive pharmaceutical compositions for producing a pharmaceutical form.

### IMPLEMENTATION OF THE INVENTION

The invention relates to a pharmaceutical composition, preferably in the form of a powder, comprising a mixture of at least one cationic, water-soluble (meth)acrylate copolymer, at least one water-insoluble polymer and at least one active ingredient having a solubility in demineralized water of 3.3 g/l or less,
characterized in that
   the water-insoluble polymer and the active ingredient are present in a ratio of at most 3.5 to 1 parts by weight, and the pharmaceutical composition has the property of releasing the active ingredient present in a medium buffered to pH 1.2 (SGFsp, Simulated Gastric Fluid sine pancreatin) in dissolved form in a concentration which, after 2 hours at pH 1.2, corresponds to at least sixteen times the solubility value of the active ingredient alone at pH 1.2.

### Cationic, Water-Soluble (Meth)Acrylate Copolymers

Cationic, water-soluble (meth)acrylate copolymers are understood to mean those (meth)acrylate copolymers with cationic groups, which are water-soluble at least within a certain pH range. In general, the pharmaceutical composition comprises only one cationic, water-soluble (meth)acrylate copolymer. However, it is also possible if appropriate for two or more cationic, water-soluble (meth)acrylate copolymers to be present alongside one another or in a mixture.

The cationic, water-soluble (meth)acrylate copolymer possibly has the function of converting the active ingredient sparingly soluble in water, in the case of a melt extrusion similar to U.S. Pat. No. 6,391,338, to a state of higher solubility in the polymer mixture.

Examples of preferred cationic, water-soluble (meth)acrylate copolymers are in particular:

### EUDRAGIT® E Type

The cationic, water-soluble (meth)acrylate copolymer may be composed partly or fully of alkyl acrylates and/or alkyl methacrylates having a tertiary amino group in the alkyl radical. Suitable (meth)acrylate copolymers are known, for example, from EP 0 058 765 B1.

The cationic, water-soluble (meth)acrylate copolymer may be composed, for example, of 30 to 80% by weight of free-radically polymerized C₁- to C₄-alkyl esters of acrylic acid or of methacrylic acid, and 70 to 20% by weight of (meth)acrylate monomers having a tertiary amino group in the alkyl radical.

Suitable monomers with functional tertiary amino groups are detailed in U.S. Pat. No. 4,705,695, column 3 line 64 to column 4 line 13. Mention should be made in particular of dimethylaminoethyl acrylate, 2-dimethylaminopropyl acrylate, dimethylaminopropyl methacrylate, dimethylaminobenzyl acrylate, dimethylaminobenzyl methacrylate, (3-dimethylamino-2,2-dimethyl)propyl acrylate, dimethylamino-2,2-dimethyl)propyl methacrylate, (3-diethylamino-2,2-dimethyl)propyl acrylate and diethylamino-2,2-dimethyl)propyl methacrylate. Particular preference is given to dimethylaminoethyl methacrylate.

The content of the monomers with tertiary amino groups in the copolymer may advantageously be between 20 and 70% by weight, preferably between 40 and 60% by weight. The proportion of the C₁- to C₄-alkyl esters of acrylic acid or methacrylic acid is 70-30% by weight. Mention should be made of methyl methacrylate, ethyl methacrylate, butyl methacrylate, methyl acrylate, ethyl acrylate and butyl acrylate.

A suitable (meth)acrylate copolymer with tertiary amino groups may be formed, for example, from 20-30% by weight of methyl methacrylate, 20-30% by weight of butyl methacrylate and 60-40% by weight of dimethylaminoethyl methacrylate.

A specifically suitable commercial (meth)acrylate copolymer with tertiary amino groups is, for example, formed from 25% by weight of methyl methacrylate, 25% by weight of butyl methacrylate and 50% by weight of dimethylaminoethyl methacrylate (EUDRAGIT® E100 or EUDRAGIT® E PO (powder form)). EUDRAGIT® E100 and EUDRAGIT® E PO are water-soluble below approx. pH 5.0 and are thus also gastric juice-soluble.

### Water-Insoluble Polymers

Water-insoluble polymers are understood to mean those polymers which are water-insoluble over the entire pH range of 1 to 14 and only swellable in water. In general, only one water-insoluble polymer is present in the pharmaceutical composition. However, it is also possible if appropriate for two or more water-insoluble polymers to be present alongside one another or in a mixture.

The water-insoluble polymer is suspected to have the function of stabilizing the active ingredient sparingly soluble in water in the state of higher solubility after release from the pharmaceutical composition over a prolonged period, and thus of slowing or preventing solubility-reducing aggregation, recrystallization or precipitation.

Examples of preferred water-insoluble polymers are in particular neutral (meth)acrylate copolymers and (meth)acrylate copolymers with quaternary amino groups:

Neutral (meth)acrylate Copolymers (EUDRAGIT® NE Type or Eudragit® NM Type)

Neutral or essentially neutral methacrylate copolymers consist at least to an extent of 95% by weight, in particular to an extent of at least 98% by weight, preferably to an extent of at least 99% by weight, in particular to an extent of at least 99% by weight, more preferably to an extent of 100% by weight, of (meth)acrylate monomers with neutral radicals, especially C₁- to C₄-alkyl radicals.

Suitable (meth)acrylate monomers with neutral radicals are, for example, methyl methacrylate, ethyl methacrylate, butyl methacrylate, methyl acrylate, ethyl acrylate, butyl acrylate. Preference is given to methyl methacrylate, ethyl acrylate and methyl acrylate.

Methacrylate monomers with anionic radicals, for example acrylic acid and/or methacrylic acid, may be present in small amounts of less than 5% by weight, preferably not more than 2% by weight, more preferably not more than 1 or 0.05 to 1% by weight.

Suitable examples are neutral or virtually neutral (meth)acrylate copolymers composed of 20 to 40% by weight of ethyl acrylate, 60 to 80% by weight of methyl methacrylate and 0 to less than 5% by weight, preferably 0 to 2 or 0.05 to 1% by weight (EUDRAGIT® NE type).

EUDRAGIT® NE and Eudragit® NM are copolymers of 30% by weight of ethyl acrylate and 70% by weight of methyl methacrylate.

Preference is given to neutral or essentially neutral methyl acrylate copolymers which, according to WO 01/68767, have been prepared as dispersions using 1-10% by weight of a nonionic emulsifier having an HLB value of 15.2 to 17.3. The latter offer the advantage that there is no phase separation with formation of crystal structures by the emulsifier (Eudragit® NM).

According to EP 1 571 164 A2, corresponding, virtually neutral (meth)acrylate copolymers with small proportions of 0.05 to 1% by weight of monoolefinically unsaturated C3-C8-carboxylic acids can, however, also be prepared by emulsion polymerization in the presence of comparatively small amounts of anionic emulsifiers, for example 0.001 to 1% by weight.

(Meth)Acrylate Copolymers with Quaternary Ammonium Groups (EUDRAGIT® RS/RL Type)

Further suitable water-insoluble (meth)acrylate copolymers are known, for example, from EP-A 181 515 or from DE-C1 617 751. Irrespective of the pH, they are water-insoluble respectively in water only swellable polymers, which are suitable for medicament coatings. One possible preparation process is bulk polymerization in the presence of a free-radical-forming initiator dissolved in the monomer mixture. Equally, the addition polymer can also be prepared by means of solution or precipitation polymerization. The addition polymer can be obtained in this way in the form of a fine powder, which is achievable in the case of bulk polymerization by grinding, and in the case of solution and precipitation polymerization, for example, by spray-drying.

A suitable water-insoluble (meth)acrylate copolymer is composed of 85 to 98% by weight of free-radically polymerized C₁- to C₄₋alkyl esters of acrylic acid or of methacrylic acid and 15 to 2% by weight of (meth)acrylate monomers having a quaternary amino group in the alkyl radical.

Preferred C₁- to C₄₋alkyl esters of acrylic acid or of methacrylic acid are methyl acrylate, ethyl acrylate, butyl acrylate, butyl methacrylate and methyl methacrylate.

A particularly preferred (meth)acrylate monomer with quaternary ammonium groups is 2-trimethylammonioethyl methacrylate chloride.

A corresponding copolymer can be formed, for example, from 50-70% by weight of methyl methacrylate, 20-40% by weight of ethyl acrylate and 7-2% by weight of 2-trimethylammonioethyl methacrylate chloride.

A specifically suitable copolymer contains 65% by weight of methyl methacrylate, 30% by weight of ethyl acrylate and 5% by weight of 2-trimethylammonioethyl methacrylate chloride (EUDRAGIT® RS).

A further suitable (meth)acrylate copolymer may be formed, for example, from 85 to less than 93% by weight of C1- to C4-alkyl esters of acrylic acid or of methacrylic acid and more than 7 to 15% by weight of (meth)acrylate monomers with a quaternary ammonium group in the alkyl radical. Such (meth)acrylate monomers are commercially available and have been used for some time for retarding coatings.

A specifically suitable copolymer contains, for example, 60% by weight of methyl methacrylate, 30% by weight of ethyl acrylate and 10% by weight of 2-trimethylammonioethyl methacrylate chloride (EUDRAGIT® RL).

In particular, useful mixtures of the (meth)acrylate copolymers mentioned also include in particular mixtures of EUDRAGIT® RS and EUDRAGIT® RL, for example in the ratio of 9:1 to 1:9 parts by weight.

### Polyvinyl Acetate/Polyvinyl Acetate Copolymers, Ethyl- and Methylcellulose

The pharmaceutical composition may also comprise, as water-insoluble polymer, a polyvinyl acetate, a polyvinyl acetate copolymer (for example Kollicoat® SR 30D or Kollidon® SR type), an ethylcellulose or a methylcellulose.

### Proportions

The water-soluble (meth)acrylate copolymer or copolymers and the water-insoluble polymer or polymers in the pharmaceutical composition may be present in a ratio relative to one another of 40:60 to 99:1 parts by weight, preferably in a ratio relative to one another of 50:50 to 95:5 parts by weight, in particular in a ratio relative to one another of 70:30 to 92:8 parts by weight. Surprisingly, even small additions of the water-insoluble polymer to the water-soluble (meth)acrylate copolymer are sufficient to achieve the inventive effect.

The proportion of the water-insoluble polymer, based on the active ingredient having a solubility in demineralized water of 3.3 g/l or less, should not be too high, since the desired improvement in solubility after 120 min at pH 1.2 by at least 16 times is otherwise not achieved.

The water-insoluble polymer and the active ingredient should be present in a ratio of at most 3.5 parts by weight of water-insoluble polymer to 1 part by weight of active ingredient, preferably of at most 3.5:1 to 0.25:1 parts by weight, in particular of at most 2.5:1 to 0.25:1 parts by weight.

In the case of the presence of a plurality of water-insoluble polymers and/or a plurality of active ingredients alongside one another, the proportions are each based on their sum.

### Active Ingredients

The pharmaceutical composition comprises at least one, generally only one, active ingredient, but if appropriate also combinations of two or more active ingredients. The active ingredient present may therefore consist of a single active ingredient or if appropriate also of a plurality of individual active ingredients.

The active ingredient(s) has/have a solubility in demineralized water of 3.3 g/l or less, preferably 2.2 g/l or less, in particular 1.1 g/l or less.

The active ingredient(s) may belong, for example, to the group of BCS classes II and IV (Biopharmaceutical classification system according to Prof. Amidon; Amidon et al., Pharm. Res. 12, 413-420 (1995)) and/or from the group of the antiandrogenics, antidepressives, antidiabetics, antirheumatics, glucocorticoids, cytostatics, migraine drugs, neuroleptics, antibiotics, oestrogens, vitamins, psychotropic drugs, ACE inhibitors, β-blockers, calcium channel blockers, diuretics, cardiac glycosides, antiepileptics, diuretics/antiglaucoma, uricostatics, H₂ receptor blockers and virostatics.

The active ingredients of BCS class II and IV have a solubility in demineralized water of 3.3 g/l or less. The active ingredients of BCS class II have good permeability, those of BCS class IV low permeability. The advantages of the invention are therefore displayed in particular for the active ingredients of BCS class II, since the availability of the active ingredient in solution here constitutes the sole limitation of its bioavailability. However, increased availability of the active ingredient in solution can also be helpful in the case of active ingredients of BCS class IV, in order to achieve a certain improvement in the bioavailability at least gradually in spite of the limitation of poor absorption into the cells (permeability) of these active ingredients.

It is possible, for example, for the active ingredient(s) bicalutamide, anastrozole, albendazole, amitryptiline, artemether, chlorpromazine, ciprofloxacin, clofazimine, dapsone, diloxanide, efavirenz, folic acid, furosemide, glibenclamide, griseofulvin, haloperidol, ivermectin, ibuprofen, idinavir, lopinavir, lumefantrin, mebendazole, mefloquin, niclosamide, nelfinavir, nifedipine, nitrofurantoin, phenyloin, pyrantel, pyremethamine, retinol, ritonavir, spironolactone, sulfadiazine, sulfasalazine, sulfamethoxazole, triclabendazole, trimethoprim, valproic acid, verapamil, warfarin, nalidixic acid, nevirapine, praziquantel, rifampicin, glimipiride, nilutamide, bromocriptine, ketotifen, letrozole, naratriptan, ganciclovir, orlistat, misoprostol, granistron, pioglitazone, lamivudine, rosiglitazone, zidovudine, enalapril, atenolol, nadolol, felodipine, bepridil, digoxin, digitoxin, carbamazepine, acetazolamide, allopurinol, cimetidine, ranitidine or oxcarbazepine to be present.

### Solubility in Water

The invention relates to active ingredients having a solubility in demineralized water of 3.3 g/l or less, preferably 3.3 g/l or less, in particular 1.1 g/l or less.

The solubility in water for the active ingredient can be defined according to DAB 10 (Deutsches Arzneibuch [German Pharmacopoeia], 10th edition with 3rd revision 1994, Deutscher Apothekerverlag, Stuttgart and Govi Verlag, Frankfurt am Main, 2nd revision (1993), IV Allgemeine Vorschriften [IV General methods], p. 5-6, “Löslichkeit und Lösungsmittel” [“Solubility and solvents”]; see also Ph. Eur. 4.07, 2004).

### Solubility at pH 1.2

The solubility at pH 1.2, i.e. the amount of active ingredient present in dissolved form, can be determined, for example, chromatographically and/or spectrometrically in a medium buffered to pH 1.2 (SGFsp, Simulated Gastric Fluid sine pancreatin) according to USP (paddle method, 100 rpm). The values of the active ingredient formulated in accordance with the invention and of the unformulated active ingredient after 120 min are compared. This simulates the conditions of an average stomach passage time. In this comparison, the solubility of the active ingredient formulated in accordance with the invention should be increased by at least 16 times, preferably by at least 18 times, in particular by at least 20 times.

The methodology according to USP, paddle method is sufficiently well known to those skilled in the art (see, for example, USP 28-NF23, General Chapter <711>, Dissolution, Apparatus 2 (paddle), Method <724>“Delayed Release (Enteric Coated) Articles-General General Drug Release Standard”, Method B (100 rpm, 37° C.)

### Process for Producing the Pharmaceutical Composition

### “Solvent Process”

The invention further relates to a

Process for preparing a pharmaceutical composition in the form of a solid with the property of releasing the active ingredient present in a medium buffered to pH 1.2 in dissolved form in a concentration which, after 2 hours at pH 1.2, corresponds to at least sixteen times the solubility value of the active ingredient alone at pH 1.2, characterized in that a solution in an organic solvent or a solvent mixture composed of the cationic, water-soluble (meth)acrylate copolymer, the active ingredient and the water-insoluble polymer is first obtained, the solvent is then removed, for example, by evaporation or applying reduced pressure, for example, by freeze-drying or spray-drying, which affords a solid with the properties mentioned.

The organic solvent may if appropriate also be a solvent mixture with other organic solvents and/or water. When water is present, the content must only be so high that, in spite of it, all constituents, the two polymer types and the active ingredient, still go into a solution. Suitable solvents are, for example, acetone, isopropanol or ethanol or mixtures thereof. A suitable example is an isopropanol/acetone mixture with 6:4 parts by weight. Suitable examples are also ethanol/water mixtures, preferably with not more than 50% by weight of water.

The process makes use of the fact that the active ingredient is sparingly soluble in water and can therefore be dissolved comparatively efficiently in an organic solvent. The cationic, water-soluble (meth)acrylate copolymers are equally also dissolvable in an organic solvent. For example, the polymers of the EUDRAGIT® E type are also commercially available in suitable form in the form of organic solutions with solids content 12.5%. The water-insoluble polymer is in turn readily soluble in an organic solvent. It is therefore possible to prepare a solution of all three components, in which case the active ingredient retains the dissolved state even after the removal of the solvent in the solid. For unknown reasons, the content of the water-insoluble polymer in the mixture has the effect that the original solubility of the active ingredient does not decline again below the threshold value after release in an intestinal juice-like medium at pH 7.2, said threshold value corresponding to at least twice the solubility value of the active ingredient in demineralized water after 4 hours.

The solvent process has the advantage of being easy to implement.

### “Melt Extrusion Process”

The melt extrusion process is preferred over the solvent process, one reason being that the handling of solvents, which is problematic for procedural, health protection and environmental protection reasons, is dispensed with.

According to the invention, the invention relates to a

process for preparing a pharmaceutical composition in the form of an extrudate with the property of releasing the active ingredient present in a medium buffered to pH 1.2 in dissolved form in a concentration which, after 2 hours at pH 1.2, corresponds to at least sixteen times the solubility value of the active ingredient alone at pH 1.2, characterized in that the cationic, water-soluble (meth)acrylate copolymer, the active ingredient and the water-insoluble polymer are mixed and melt-extruded at a temperature in the range of 60 to 220° C., preferably of 80 to 180° C.

The melt extrusion process can be performed with the aid of an extruder, especially by means of a twin-screw extruder. It is favourable when the extruder or the twin-screw extruder is equipped with a degassing zone. The cationic, water-soluble and the water-insoluble polymer can be incorporated as a solid, as a polymer solution or as a polymer dispersion. The active ingredient can be added as a solid, as a solution or as a suspension. The extrudate is preferably processed by means of strand granulation and hot-cut methods to give cylindrical, elongated strand granules, or by hot-cutting with cooling to give rounded pellets. EP 1 563 987 A1 describes a suitable apparatus for producing rounded pellets (pelletizer). Granules can preferably be ground to powders with, for example, particle sizes of less than/equal to 1 mm, preferably in the range of 50 to 500 μm.

### Process for Producing a Pharmaceutical Form

The invention further relates to a

process for producing an inventive pharmaceutical form comprising an inventive pharmaceutical composition, characterized in that a pharmaceutical composition is prepared by the above-described solvent process or the melt extrusion process, processed further to granules, pellets or powders, if appropriate formulated by means of pharmaceutically customary excipients, and processed in a manner known per se, for example by mixing, compressing, powder layering and/or encapsulation to a pharmaceutical form, for example to tablets, or preferably to a multiparticulate pharmaceutical form, especially to pellet-containing tablets, minitablets, capsules, sachets or reconstitutable powders.

### Production of Tablets and Multiparticulate Pharmaceutical Forms

The inventive pharmaceutical composition is suitable in particular for producing pharmaceutical forms in tablet form and for use in multiparticulate pharmaceutical forms. The inventive pharmaceutical composition is preferably present in the form of a powder and can be used directly in virtually all known pharmaceutical formulations in which the active ingredient is incorporated in powder form instead of the active ingredient. In this way, for example, as in WO 01/68058 or WO 2005/046649, neutral cores (non-pareilles) can be coated with the pharmaceutical composition in powder form and a binder in the powder layering process. Subsequently, the coated cores are formulated to finished pharmaceutical forms with further excipients and polymer layers, as prescribed in WO 01/68058 or WO 2005/046649.

For the multiparticulate pharmaceutical form, the pharmaceutical composition in the form of a powder, even without neutral core, can be processed with binders by rounding, compression to active ingredient-containing particles or pellets which may themselves be provided with appropriate polymeric coating layers to control the active ingredient release.

The production of multiparticulate pharmaceutical forms to give tablets by compression of a pharmaceutically customary binder with active ingredient-containing particles is described in detail, for example, Beckert et al. (1996), “Compression of enteric-coated pellets to disintegrating tablets”, International Journal of Pharmaceutics 143, p. 13-23 and in WO 96/01624.

Film coatings on active ingredient-containing pellets are typically applied in fluidized bed systems. Film formers are typically mixed with plasticizers and release agents by a suitable process. In this process, the film formers may be present as a solution or suspension. The excipients for the film formation may likewise be dissolved or suspended. Organic or aqueous solvents or dispersants can be used. To stabilize the dispersion, stabilizers can additionally be used (example: Tween 80 or other suitable emulsifiers or stabilizers).

Examples of release agents are glyceryl monostearate or other suitable fatty acid derivatives, silica derivatives or talc. Examples of plasticizers are propylene glycol, phthalates, polyethylene glycols, sebacates or citrates, and also other substances mentioned in the literature.

A separating layer, which serves for the separation of active ingredient and coating material for the purposes of preventing interactions, can be applied between active ingredient-containing layer and an intestinal juice-soluble copolymer layer which is optionally present. This layer can consist of inert film formers (for example HPMC or HPC) or, for example, talc or other suitable pharmaceutical substances. It is equally possible to use combinations of film formers and talc or similar substances.

It is also possible to apply a separating layer composed of partly or fully neutralized copolymer dispersions which may, for example, comprise anionic (meth)acrylate copolymers.

Mixtures for producing tablets from coated particles are prepared by mixing the pellets with suitable binders for the tabletting, if necessary the addition of disintegration-promoting substances and if necessary the addition of lubricants. The mixing can take place in suitable machines. Unsuitable mixers are those which lead to damage to the coated particles, for example ploughshare mixers. To achieve suitable short disintegration times, a specific sequence may be required in the addition of the excipients to the coated particles. Premixing with the coated particles comprising the lubricant or mould release agent magnesium stearate allows its surface to be hydrophobicized and thus adhering to be prevented.

Mixtures suitable for tabletting contain typically 3 to 15% by weight of a disintegration assistant, for example Kollidon CL, and, for example, 0.1 to 1% by weight of a lubricant and mould release agent such as magnesium stearate. The binder content is determined by the required proportion of coated particles.

Typical binders are, for example, Cellactose®, microcrystalline cellulose, calcium phosphates, Ludipress®, lactose or other suitable sugars, calcium sulphates or starch derivatives. Preference is given to substances of low bulk density.

Typical disintegration assistants (disintegrants) are crosslinked starch or cellulose derivatives, and also crosslinked polyvinylpyrrolidone. Cellulose derivatives are equally suitable. Selection of a suitable binder allows the use of disintegration assistants to be dispensed with.

Typical lubricants and mould release agents are magnesium stearates or other suitable salts of fatty acids or substances mentioned in the literature for this purpose (for example lauric acid, calcium stearate, talc, etc.). When suitable machines (for example tabletting press with external lubrication) or suitable formulations are used, the use of a lubricant and mould release agent in the mixture can be dispensed with.

An excipient for flow improvement can optionally be added to the mixture (for example high-dispersion silica derivatives, talc, etc.).

The tabletting can be effected on customary tabletting presses, excentric presses or rotary tabletting presses, at pressing forces in the range of 5 to 40 kN, preferably 10-20 kN. The tabletting presses can be equipped with systems for external lubrication.

### Excipients

Typical excipients or additives are preferably added in a manner known per se to the inventive composition in the course of production of the granules, pellets or powder. All excipients used must of course fundamentally be toxicologically uncontroversial and especially be usable in medicaments without risk for patients.

Use amounts and use of the customary additives in medicament coatings are familiar to those skilled in the art. Typical additives may, for example, be release agents, pigments, stabilizers, antioxidants, pore formers, penetration promoters, glosses, aromas or flavourings. They serve as processing excipients and should ensure a reliable and reproducible production process and good long-term storage stability, or they achieve additional advantageous properties in the pharmaceutical form. They are added to the polymer preparations before the processing and can influence the permeability of the coatings, which can if appropriate be utilized as an additional control parameter.

Release Agents:

Release agents generally have lipophilic properties and are generally added to the spray suspensions. They prevent agglomeration of the cores during the filming. Preference is given to using talc, magnesium stearate or calcium stearate, ground silica, kaolin or nonionic emulsifiers having an HLB value between 3 and 8. Typical use amounts for release agents are between 0.5 to 100% by weight based on the sum of active ingredient, water-soluble (meth)acrylate copolymer and water-insoluble polymer.

Pigments:

The pigments to be used nontoxic and suitable for pharmaceutical purposes. On this subject, see also, for example: Deutsche Forschungsgemeinschaft [German Research Institute], Farbstoffe für Lebensmittel [Dyes for Foods], Harald Boldt Verlag KG, Boppard (1978); Deutsche Lebensmittelrundschau 74, 4, p. 156 (1978); German Medicament Dyes Act of 25 Aug. 1980.

Suitable pigments are, for example, aluminium oxide pigments or orange yellow, cochineal red lake, chromatic pigments based on aluminium oxide or azo dyes, sulphonic acid dyes, Orange Yellow S (E110, C.I. 15985, FD&C Yellow 6), Indigo Carmine (E132, C.I. 73015, FD&C Blue 2), Tartrazine (E 102, C.I. 19140, FD&C Yellow 5), Ponceau 4R (E 125, C.I. 16255, FD&C Cochineal Red A), Quinoline Yellow (E 104, C.I. 47005, FD&C Yellow 10), Erythrosin (E127, C.I. 45430, FD&C Red 3), Azorubin (E 122, C.I. 14720, FD&C Carmoisine), Amaranth (E 123, C.I. 16185, FD&C Red 2), Brilliant Acid Green (E 142, C.I. 44090, FD&C Green S).

The reported E numbers of the pigments are based on the EU numbering. On this subject, see also “Deutsche Forschungsgemeinschaft, Farbstoffe für Lebensmittel, Harald Boldt Verlag KG, Boppard (1978); Deutsche Lebensmittelrundschau 74, 4, p. 156 (1978);

German Medicament Dyes Act of 25 Aug. 1980. The FD&C numbers are based on the approval in Food, Drugs and Cosmetics by U.S. Food and Drug Administration (FDA), described in: U.S. Food and Drug Administration, Center for Food Safety and Applied Nutrition, Office of Cosmetics and Colors: Code of Federal Regulations—Title 21 Color Additive Regulations Part 82, Listing of Certified Provisionally Listed Colors and Specifications (CFR 21 Part 82).

### Plasticizers

Further additives may also be plasticizers. Typical amounts are between 0 and 50% by weight, preferably from 5 to 20% by weight.

Depending on the type (lipophilic or hydrophilic) and amount added, plasticizers may influence the functionality of the polymer layer. By virtue of physical interaction with the polymer, plasticizers achieve a lowering of the glass transition temperature and, depending on the amount added, promote filming. Suitable substances generally have a molecular weight between 100 and 20,000 and contain one or more hydrophilic groups in the molecule, for example hydroxyl, ester or amino groups.

Examples of suitable plasticizers are alkyl citrates, glyceryl esters, alkyl phthalates, alkyl sebacates, sucrose esters, sorbitan esters, diethyl sebacate, dibutyl sebacate and polyethylene glycols 200 to 12,000. Preferred plasticizers are triethyl citrate (TEC), acetyltriethyl citrate (ATEC) and dibutyl sebacate (DBS). Mention should also be made of esters generally liquid at room temperature, such as citrates, phthalates, sebacates or castor oil. Preference is given to using citric and sebacic esters.

The addition of plasticizers to the formulation can be undertaken in a known manner, directly, in aqueous solution or after thermal pretreatment of a mixture. It is also possible to use mixtures of plasticizers.

### Pharmaceutical Form

The invention further relates to a pharmaceutical form comprising an inventive pharmaceutical composition.

### Use

The invention further relates to the use of the inventive pharmaceutical composition for producing a pharmaceutical form. The pharmaceutical composition may preferably be incorporated in powder form instead of a pulverulent active ingredient. In the inventive formulation, the powder has the property of releasing the active ingredient present in a medium buffered to pH 1.2 in dissolved form in a concentration which, after 2 hours at pH 1.2, corresponds to at least sixteen times the solubility value of the active ingredient alone at pH 1.2. This makes it possible to introduce active ingredients which are sparingly soluble per se into pharmaceutical forms of all types in a state of elevated solubility.

### ADVANTAGEOUS EFFECTS OF THE INVENTION

An advantageous effect of the inventive pharmaceutical composition is in particular that active ingredients sparingly soluble in water are converted to a state of higher solubility, this state, in delimitation from the prior art (see Example 1), remaining stable at pH 1.2 over a period of 120 min. The period of 120 min at pH 1.2 simulates an average stomach passage time. In this way, it is possible, after initial elevated solubility of the active ingredient, to reduce or even to prevent its recrystallization in the course of the stomach residence time. This increases the bioavailability as a function of time, and in particular considerably at the moment of transfer in the intestinal tract.

The gastric juice-like environment represents a high test requirement, so that it can be assumed that the state of elevated solubility, when it is attained stably in the test at pH 1.2 after 120 min, no longer significantly changes disadvantageously even after transfer into the section of the intestine at the higher pH values which exist there.

The inventive pharmaceutical composition is therefore suitable not only for active ingredients which are to be released in the stomach but virtually also for all other pharmaceutical forms, for example for gastric resistant coated pharmaceutical forms and/or pharmaceutical forms with a retarding formulation which release the active ingredient actually within the stomach. This makes it possible in a better manner than to date also to attain therapeutically required, comparatively high blood levels even in the case of active ingredients sparingly soluble in water, and also to maintain them over prolonged periods.

The pharmaceutical composition is preferably present in powder form and can be used virtually in all formulations in which the active ingredient is processed in powder form in its place. Owing to the elevated solubility, new possible therapies are in principle opened up in this way.

The advantageous effects of the invention can be explained, for example, with reference to the examples.

### EXAMPLES

### A) Polymers

EUDRAGIT® E is a water-soluble copolymer of 25% by weight of methyl methacrylate, 25% by weight of butyl methacrylate and 50% by weight of dimethylaminoethyl methacrylate.

EUDRAGIT® NE is a water-insoluble copolymer of 30% by weight of ethyl acrylate and 70% by weight of methyl methacrylate.

EUDRAGIT® RL is a water-insoluble copolymer 60% by weight of methyl methacrylate, 30% by weight of ethyl acrylate and 10% by weight of 2-trimethylammonioethyl methacrylate chloride.

Kollicoat® SR is a water-insoluble polymer (a polyvinyl acetate copolymer).

Kollicoat® IR (a vinyl acetate-ethylene glycol block copolymer) is a water-soluble polymer.

PEG 6000: polyethylene glycol 6000 (water-soluble polymer).

### B) Production of the Extrudates for Examples 1 to 14

The samples are produced by melt extrusion on a twin-screw extruder (Leistritz MICRO 18 GL 40 D Pharma). The temperature was selected such that at least one zone is above the melting point of the active ingredient. The extrusion was performed in a range between 70-170° C.

Felodipine, the water-soluble (meth)acrylate copolymer EUDRAGIT® E and, if appropriate, the “second” polymer are metered in by means of solid or liquid metering devices, mixed in the extruder, melted and extruded. In the inventive examples, the “second” polymer is water-insoluble and is present in a ratio relative to the active ingredient of at most 3.5:1. The speed was 200-250 rpm. The resulting melt is drawn off by means of an air-cooled draw belt and then comminuted in a strand granulator. Subsequently, the granule is ground at 6000 l/min in a Retsch ultracentrifugal mill with a 250 μm screen insert and then (<250 μm) screened.

### Composition of the Individual Extrudates in % by Weight

TABLE 1

| | | EUDRAGIT ® | EUDRAGIT ® | Kollicoat ® | EUDRAGIT ® | PEG | Kollicoat ® |
|---|---|---|---|---|---|---|---|
| Example | Felodipine | E | NE | SR | RL | 6000 | IR |
| 1 | 10.0 | 90.0 | | | | | |
| 2 | 9.5 | 85.7 | 4.8 | | | | |
| 3 | 9.1 | 81.8 | 9.1 | | | | |
| 4 | 8.3 | 75.0 | 16.7 | | | | |
| 5 | 7.1 | 64.3 | 28.6 | | | | |
| 6 | 6.3 | 56.3 | 37.5 | | | | |
| 7 | 9.1 | 81.8 | | 9.1 | | | |
| 8 | 8.3 | 75.0 | | 16.7 | | | |
| 9 | 9.1 | 81.8 | | | 9.1 | | |
| 10 | 8.3 | 75.0 | | | 16.7 | | |
| 11 | 9.1 | 81.8 | | | | 9.1 | |
| 12 | 8.3 | 75.0 | | | | 16.7 | |
| 13 | 9.1 | 81.8 | | | | | 9.1 |
| 14 | 8.3 | 75.0 | | | | | 16.7 |

Inventive: Examples 2-4, 7-10
Noninventive: Examples 1, 5, 6, 11-14

### Examples 1 to 14

### Release of the Active Ingredient Felodipine from the Ground Granules

The release of the active ingredient from the ground granules was performed in a paddle apparatus (DT 700 Dissolution tester, Erweka) USP 26 method 2. The samples were weighed in corresponding in each case to 10 mg of felodipine. 500 ml of SGFsp (simulated gastric fluid sine pancreatin, USP) pH 1.2 (37° C.±0.5) were used as the medium and the stirrer speed was 100 rpm. 5 ml samples were taken at certain intervals, filtered through a membrane filter (Rezist® 30/0.45 μm PTFE, Schleicher & Schüll), and diluted 1:1 with methanol. The first 2 ml were discarded. The volume withdrawn was replaced with fresh, temperature-controlled medium. The amount of felodipine released was detected by means of HPLC.

(Column used: RP 18 (Lichrospher 100.5 μm, 125×4, Merck), eluent: acetonitrile:methanol:phosphate buffer pH 3, flow rate: 1 ml/min, wavelength: 362 nm).

### Determination of the Solubility of Felodipine at pH 1.2:

Felodipine has a solubility in water of <1 mg/l (0.0001 g/l). The solubility for felodipine was determined for comparison in a medium buffered to pH 1.2 (SGFsp pH 1.2). For this purpose, 10 mg were kept in motion at 37° C. on an orbital shaker in 20 ml of medium for 24 hours. The concentration was determined by means of HPLC. For felodipine alone, without inventive formulation, a solubility of 0.5 mg/l was determined.

The tables for Examples 1 to 14 reproduce the solubility values of felodipine at pH 1.2 as a function of time. Three parallel experiments (vessels 1 to 3) were performed.

### Example 1

### Noninventive

Extrudate composed of 90/10 EUDRAGIT ® E
and felodipine (noninventive)

| | Vessel 1 | Vessel 2 | Vessel 3 | |
|---|---|---|---|---|
| Time | (g/l) | (g/l) | (g/l) | Mean |
| 0 | 0.0000 | 0.0000 | 0.0000 | 0.0000 |
| 5 | 0.0126 | 0.0142 | 0.0164 | 0.0144 |
| 10 | 0.0129 | 0.0139 | 0.0158 | 0.0142 |
| 15 | 0.0111 | 0.0123 | 0.0144 | 0.0126 |
| 30 | 0.0073 | 0.0086 | 0.0107 | 0.0089 |
| 45 | 0.0055 | 0.0077 | 0.0077 | 0.0068 |
| 60 | 0.0048 | 0.0051 | 0.0059 | 0.0052 |
| 90 | 0.0042 | 0.0046 | 0.0043 | 0.0044 |
| 120 | 0.0037 | 0.0037 | 0.0038 | 0.0037 |

The solubility maximum here is attained after 5 min, but then falls significantly.

### Example 2

Extrudate composed of 9.5/85.7/4.8 felodipine,
EUDRAGIT ® E and EUDRAGIT ® NE

| | Vessel 1 | Vessel 2 | Vessel 3 | |
|---|---|---|---|---|
| Time | (g/l) | (g/l) | (g/l) | Mean |
| 0 | 0 | 0 | 0 | 0 |
| 5 | 0.0120 | 0.0160 | 0.0159 | 0.0146 |
| 10 | 0.0141 | 0.0163 | 0.0168 | 0.0157 |
| 15 | 0.0154 | 0.0163 | 0.0164 | 0.0160 |
| 30 | 0.0160 | 0.0162 | 0.0155 | 0.0159 |
| 45 | 0.0158 | 0.0156 | 0.0152 | 0.0155 |
| 60 | 0.0154 | 0.0167 | 0.0153 | 0.0158 |
| 90 | 0.0150 | 0.0152 | 0.0153 | 0.0151 |
| 120 | 0.0150 | 0.0145 | 0.0141 | 0.0146 |

### Example 3

Extrudate composed of 9.1/81.8/9.1 felodipine,
EUDRAGIT ® E and EUDRAGIT ® NE

| | Vessel 1 | Vessel 2 | Vessel 3 | |
|---|---|---|---|---|
| Time | (g/l) | (g/l) | (g/l) | Mean |
| 0 | 0 | 0 | 0 | 0 |
| 5 | 0.0099 | 0.0113 | 0.0121 | 0.0111 |
| 10 | 0.0127 | 0.0122 | 0.0123 | 0.0124 |
| 15 | 0.0128 | 0.0119 | 0.0120 | 0.0122 |
| 30 | 0.0121 | 0.0117 | 0.0119 | 0.0119 |
| 45 | 0.0120 | 0.0115 | 0.0123 | 0.0119 |
| 60 | 0.0124 | 0.0114 | 0.0120 | 0.0119 |
| 90 | 0.0133 | 0.0114 | 0.0114 | 0.0120 |
| 120 | 0.0116 | 0.0112 | 0.0111 | 0.0113 |

### Example 4

Extrudate composed of 8.3/75/16.7 felodipine,
EUDRAGIT ® E and EUDRAGIT ® NE

| | Vessel 1 | Vessel 2 | Vessel 3 | |
|---|---|---|---|---|
| Time | (g/l) | (g/l) | (g/l) | Mean |
| 0 | 0.0000 | 0.0000 | 0.0000 | 0.0000 |
| 5 | 0.0044 | 0.0087 | 0.0087 | 0.0072 |
| 10 | 0.0094 | 0.0097 | 0.0099 | 0.0096 |
| 15 | 0.0097 | 0.0096 | 0.0093 | 0.0095 |
| 30 | 0.0095 | 0.0093 | 0.0094 | 0.0094 |
| 45 | 0.0098 | 0.0092 | 0.0099 | 0.0096 |
| 60 | 0.0094 | 0.0094 | 0.0096 | 0.0095 |
| 90 | 0.0095 | 0.0094 | 0.0099 | 0.0096 |
| 120 | 0.0103 | 0.0097 | 0.0095 | 0.0098 |

### Example 5

### Noninventive

Extrudate composed of 7.14/64.28/28.58 felodipine,
EUDRAGIT ® E and EUDRAGIT ® NE

| | Vessel 1 | Vessel 2 | Vessel 3 | Mean |
|---|---|---|---|---|
| Time | (g/l) | (g/l) | (g/l) | (g/l) |
| 0 | 0 | 0 | 0 | 0 |
| 5 | 0.0028 | 0.0046 | 0.0041 | 0.0039 |
| 10 | 0.0043 | 0.0044 | 0.0037 | 0.0041 |
| 15 | 0.0044 | 0.0041 | 0.0051 | 0.0045 |
| 30 | 0.0053 | 0.0053 | 0.0055 | 0.0054 |
| 45 | 0.0056 | 0.0051 | 0.0058 | 0.0055 |
| 60 | 0.0053 | 0.0056 | 0.0060 | 0.0056 |
| 120 | 0.0046 | 0.0055 | 0.0050 | 0.0051 |

### Example 6

### Noninventive

Extrudate composed of 6.25/56.25/37.5 felodipine,
EUDRAGIT ® E and EUDRAGIT ® NE

| | Vessel 1 | Vessel 2 | Vessel 3 | Mean |
|---|---|---|---|---|
| Time | (g/l) | (g/l) | (g/l) | (g/l) |
| 0 | 0 | 0 | 0 | 0 |
| 5 | 0.0008 | 0.0024 | 0.0024 | 0.0019 |
| 10 | 0.0022 | 0.0028 | 0.0027 | 0.0026 |
| 15 | 0.0026 | 0.0032 | 0.0028 | 0.0029 |
| 30 | 0.0036 | 0.0033 | 0.0030 | 0.0033 |
| 45 | 0.0031 | 0.0034 | 0.0032 | 0.0033 |
| 60 | 0.0037 | 0.0038 | 0.0034 | 0.0036 |
| 120 | 0.0011 | 0.0023 | 0.0010 | 0.0015 |

### Example 7

Extrudate composed of 9.1/81.8/9.1 felodipine,
EUDRAGIT ® E and Kollicoat ® SR

| | Vessel 1 | Vessel 2 | Vessel 3 | Mean |
|---|---|---|---|---|
| Time | (g/l) | (g/l) | (g/l) | (g/l) |
| 0 | 0.0000 | 0.0000 | 0.0000 | 0.0000 |
| 5 | 0.0090 | 0.0116 | 0.0116 | 0.0107 |
| 10 | 0.0109 | 0.0119 | 0.0126 | 0.0118 |
| 15 | 0.0113 | 0.0119 | 0.0122 | 0.0118 |
| 30 | 0.0114 | 0.0116 | 0.0112 | 0.0114 |
| 45 | 0.0113 | 0.0116 | 0.0118 | 0.0116 |
| 60 | 0.0116 | 0.0123 | 0.0117 | 0.0119 |
| 120 | 0.0110 | 0.0119 | 0.0117 | 0.0115 |

### Example 8

Extrudate composed of 8.3/75/16.7 felodipine,
EUDRAGIT ® E and Kollicoat ® SR

| | Vessel 1 | Vessel 2 | Vessel 3 | Mean |
|---|---|---|---|---|
| Time | (g/l) | (g/l) | (g/l) | (g/l) |
| 0 | 0.0000 | 0.0000 | 0.0000 | 0.0000 |
| 5 | 0.0069 | 0.0102 | 0.0095 | 0.0088 |
| 10 | 0.0100 | 0.0100 | 0.0101 | 0.0100 |
| 15 | 0.0117 | 0.0094 | 0.0101 | 0.0104 |
| 30 | 0.0096 | 0.0095 | 0.0098 | 0.0096 |
| 45 | 0.0094 | 0.0096 | 0.0103 | 0.0098 |
| 60 | 0.0096 | 0.0097 | 0.0098 | 0.0097 |
| 120 | 0.0097 | 0.0092 | 0.0099 | 0.0096 |

### Example 9

Extrudate comprising 9.1/81.8/9.1 felodipine,
EUDRAGIT ® E and EUDRAGIT ® RL

| | Vessel 1 | Vessel 2 | Vessel 3 | Mean |
|---|---|---|---|---|
| Time | (g/l) | (g/l) | (g/l) | (g/l) |
| 0 | 0 | 0 | 0 | 0 |
| 5 | 0.0112 | 0.0130 | 0.0131 | 0.0124 |
| 10 | 0.0128 | 0.0135 | 0.0139 | 0.0134 |
| 15 | 0.0136 | 0.0144 | 0.0140 | 0.0140 |
| 30 | 0.0135 | 0.0142 | 0.0138 | 0.0138 |
| 45 | 0.0134 | 0.0132 | 0.0139 | 0.0135 |
| 60 | 0.0130 | 0.0123 | 0.0130 | 0.0128 |
| 120 | 0.0117 | 0.0113 | 0.0121 | 0.0117 |

### Example 10

Extrudate comprising 8.3/75/16.7 felodipine,
EUDRAGIT ® E and EUDRAGIT ® RL

| | Vessel 1 | Vessel 2 | Vessel 3 | Mean |
|---|---|---|---|---|
| Time | (g/l) | (g/l) | (g/l) | (g/l) |
| 0 | 0.0000 | 0.0000 | 0.0000 | 0.0000 |
| 5 | 0.0077 | 0.0087 | 0.0117 | 0.0093 |
| 10 | 0.0101 | 0.0092 | 0.0101 | 0.0098 |
| 15 | 0.0103 | 0.0103 | 0.0101 | 0.0102 |
| 30 | 0.0101 | 0.0105 | 0.0103 | 0.0103 |
| 45 | 0.0101 | 0.0098 | 0.0104 | 0.0101 |
| 60 | 0.0102 | 0.0100 | 0.0097 | 0.0100 |
| 120 | 0.0095 | 0.0097 | 0.0106 | 0.0099 |

### Example 11

### Noninventive

Extrudate comprising 9.1/81.8/9.1 felodipine,
EUDRAGIT ® E and PEG 6000

| | Vessel 1 | Vessel 2 | Vessel 3 | Mean |
|---|---|---|---|---|
| Time | (g/l) | (g/l) | (g/l) | (g/l) |
| 0 | 0 | 0 | 0 | 0 |
| 5 | 0.0125 | 0.0161 | 0.0161 | 0.0149 |
| 10 | 0.0144 | 0.0158 | 0.0158 | 0.0153 |
| 15 | 0.0139 | 0.0147 | 0.0147 | 0.0144 |
| 30 | 0.0100 | 0.0106 | 0.0106 | 0.0104 |
| 45 | 0.0075 | 0.0076 | 0.0076 | 0.0075 |
| 60 | 0.0052 | 0.0058 | 0.0058 | 0.0056 |
| 120 | 0.0038 | 0.0043 | 0.0043 | 0.0041 |

### Example 12

### Noninventive

Extrudate comprising 8.3/75/16.7 felodipine,
EUDRAGIT ® E and PEG 6000

| | Vessel 1 | Vessel 2 | Vessel 3 | Mean |
|---|---|---|---|---|
| Time | (g/l) | (g/l) | (g/l) | (g/l) |
| 0 | 0 | 0 | 0 | 0 |
| 5 | 0.0152 | 0.0168 | 0.0203 | 0.0174 |
| 10 | 0.0156 | 0.0157 | 0.0145 | 0.0153 |
| 15 | 0.0144 | 0.0146 | 0.0152 | 0.0147 |
| 30 | 0.0109 | 0.0105 | 0.0092 | 0.0102 |
| 45 | 0.0087 | 0.0082 | 0.0081 | 0.0083 |
| 60 | 0.0073 | 0.0074 | 0.0072 | 0.0073 |
| 120 | 0.0062 | 0.0063 | 0.0058 | 0.0061 |

### Example 13

### Noninventive

Extrudate comprising 9.1/81.8/9.1 felodipine,
EUDRAGIT ® E and Kollicoat ® IR

| | Vessel 1 | Vessel 2 | Vessel 3 | Mean |
|---|---|---|---|---|
| Time | (g/l) | (g/l) | (g/l) | (g/l) |
| 0 | 0 | 0 | 0 | 0 |
| 5 | 0.0135 | 0.0224 | 0.0164 | 0.0174 |
| 10 | 0.0157 | 0.0180 | 0.0159 | 0.0165 |
| 15 | 0.0166 | 0.0168 | 0.0162 | 0.0165 |
| 30 | 0.0148 | 0.0151 | 0.0147 | 0.0149 |
| 45 | 0.0122 | 0.0122 | 0.0117 | 0.0120 |
| 60 | 0.0100 | 0.0105 | 0.0103 | 0.0103 |
| 120 | 0.0068 | 0.0067 | 0.0072 | 0.0069 |

### Example 14

### Noninventive

Extrudate comprising 8.3/75/16.7 felodipine,
EUDRAGIT ® E and Kollicoat ® IR

| | Vessel 1 | Vessel 2 | Vessel 3 | Mean |
|---|---|---|---|---|
| Time | (g/l) | (g/l) | (g/l) | (g/l) |
| 0 | 0 | 0 | 0 | 0 |
| 5 | 0.0104 | 0.0130 | 0.0140 | 0.0125 |
| 10 | 0.0130 | 0.0137 | 0.0139 | 0.0136 |
| 15 | 0.0137 | 0.0141 | 0.0138 | 0.0139 |
| 30 | 0.0134 | 0.0140 | 0.0138 | 0.0138 |
| 45 | 0.0111 | 0.0125 | 0.0134 | 0.0123 |
| 60 | 0.0095 | 0.0116 | 0.0115 | 0.0109 |
| 90 | 0.0072 | 0.0079 | 0.0096 | 0.0082 |
| 120 | 0.00674 | 0.0084 | 0.00768 | 0.0076 |

### Summary of Examples 1 to 14 in Relation to the Increase in Solubility of Felodipine at pH 1.2 after 120 min

The results are compiled in Table 2 below.

TABLE 2

| | | | Concentration |
|---|---|---|---|
| | | Parts by weight | increase |
| | Dissolved active | of water-insoluble | (multiple) based |
| | ingredient | polymer to 1 part | on solubility |
| | [g/l] after 120 | by weight of | of the active |
| Example | min at pH 1.2 | activeingredient | ingredient alone |
| felodipine | 0.0005 | — | — |
| alone | | | |
| 1 | 0.0037 | — | 7.4 |
| 2 | 0.0146 | 0.5 | 29.2 |
| 3 | 0.0113 | 1 | 22.6 |
| 4 | 0.0098 | 2 | 19.6 |
| 5 | 0.0050 | 4 | 10.0 |
| 6 | 0.0015 | 6 | 3.0 |
| 7 | 0.0115 | 1 | 23.0 |
| 8 | 0.0096 | 2 | 19.2 |
| 9 | 0.0117 | 1 | 23.4 |
| 10 | 0.0099 | 2 | 19.8 |
| 11 | 0.0041 | 1*) | 8.2 |
| 12 | 0.0061 | 2*) | 12.2 |
| 13 | 0.0069 | 1*) | 13.8 |
| 14 | 0.0076 | 2*) | 15.2 |

*)Ratio to the “second” polymer reported, which is, however, water-soluble in Ex. 11-14.

### Inventive Examples 2-4, 7-10

In all examples, a solubility increase after 120 min at pH 1.2 by at least 16 times the value of felodipine alone at pH 1.2 is found.

### Noninventive Examples 1, 5, 6, 11-14

### Example 1

Without the inventive addition of a water-insoluble polymer, the initially good solubility (after 5 min, see the individual values of Example 1) falls by the factor of 7.4 after 120 min.

### Examples 5 and 6

When the water-insoluble polymer is used, based on the active ingredient, in a ratio of more than 3.5 to 1 parts by weight, the solubility improvement is below 16 times that of the active ingredient alone.

### Examples 11 to 14

When, instead of a water-insoluble polymer, a water-soluble polymer is used, the solubility improvement is below 16 times that of the active ingredient alone.

1. A pharmaceutical composition comprising:
(i) at least one cationic, water-soluble (meth)acrylate copolymer, (ii) at least one water-insoluble polymer, and (iii) at least one active ingredient having a solubility in demineralized water of 3.3 g/l or less,
wherein
(i), (ii), and (iii) are present as a mixture,
the water-insoluble polymer and the active ingredient are present in a ratio of at most 3.5 to 1 parts by weight, and
the pharmaceutical composition has the property of releasing the active ingredient present in a medium buffered to pH 1.2 in dissolved form in a concentration which, after 2 hours at pH 1.2, corresponds to at least sixteen times the solubility value of the active ingredient alone at pH 1.2.

2. The pharmaceutical composition according to claim 1, wherein the cationic, water-soluble (meth)acrylate copolymer and the water-insoluble polymer are present in a ratio relative to one another of 40:60 to 99:1 parts by weight.

3. The pharmaceutical composition according to claim 1, wherein the cationic, water-soluble (meth)acrylate copolymer comprises an alkyl acrylate, an alkyl methacrylate, or a mixture thereof having a tertiary amino group in the alkyl radical.

4. The pharmaceutical composition according to claim 3, wherein the cationic, water-soluble (meth)acrylate copolymer comprising 30 to 80% by weight of C₁- to C₄₋alkyl esters of acrylic acid, and 70 to 20% by weight of (meth)acrylate monomers having a tertiary amino group in the alkyl radical.

5. The pharmaceutical composition according to claim 3, wherein the water-soluble (meth)acrylate copolymer is an addition polymer comprising 20-30% by weight of methyl methacrylate, 20-30% by weight of butyl methacrylate and 60-40% by weight of dimethylaminoethyl methacrylate.

6. The pharmaceutical composition according to claim 1, wherein the water-insoluble polymer is a copolymer comprising 20 to 40% by weight of ethyl acrylate and 60 to 80% by weight of methyl methacrylate and 0 to less than 5% by weight of acrylic acid, methacrylic acid, or a mixture thereof.

7. The pharmaceutical composition according to claim 1, wherein the water-insoluble polymer is an addition polymer comprising 98 to 88% by weight of C₁- to C₄-alkyl esters of acrylic acid or of methacrylic acid and 2 to 12% by weight of (meth)acrylate monomers having a quaternary amino group.

8. The pharmaceutical composition according to claim 1, wherein the water-insoluble polymer is selected from the group consisting of a polyvinyl acetate, a polyvinyl acetate copolymer, an ethylcellulose and a methylcellulose.

9. The pharmaceutical composition according to claim 1, wherein the active ingredient is selected from the group consisting of a biopharmaceutical belonging to BCS class II, a biopharmaceutical belonging to class IV, an antiandrogenic, an antidepressive, an antidiabetic, an antirheumatic, a glucocorticoid, a cytostatic, a migraine drug, a neuroleptic, an antibiotic, an oestrogen, a vitamin, a psychotropic drug, an ACE inhibitor, a β-blocker, a calcium channel blocker, a diuretic, a cardiac glycoside, an anti-epileptic, a diuretic/antiglaucoma, an uricostatic, an H₂ receptor blocker and a virostatic, or a mixture thereof.

10. The pharmaceutical composition according to claim 1, wherein the active ingredient is selected from the group consisting of bicalutamide, anastrozole, albendazole, amitryptiline, artemether, chlorpromazine, ciprofloxacin, clofazimine, dapsone, diloxanide, efavirenz, folic acid, furosemide, glibenclamide, griseofulvin, haloperidol, ivermectin, ibuprofen, idinavir, lopinavir, lumefantrin, mebendazole, mefloquin, niclosamide, nelfinavir, nifedipine, nitrofurantoin, phenyloin, pyrantel, pyremethamine, retinol, ritonavir, spironolactone, sulfadiazine, sulfasalazine, sulfamethoxazole, triclabendazole, trimethoprim, valproic acid, verapamil, warfarin, nalidixic acid, nevirapine, praziquantel, rifampicin, glimipiride, nilutamide, bromocriptine, ketotifen, letrozole, naratriptan, ganciclovir, orlistat, misoprostol, granistron, pioglitazone, lamivudine, rosiglitazone, zidovudine, enalapril, atenolol, nadolol, felodipine, bepridil, digoxin, digitoxin, carbamazepine, acetazolamide, allopurinol, cimetidine, ranitidine and oxcarbazepine.

11. The pharmaceutical composition according to claim 1, wherein said composition is in powder form.

12. A process for preparing a pharmaceutical composition according to claim 1, in the form of a granulated or ground extrudate comprising:
(a) mixing the cationic, water-soluble (meth)acrylate copolymer, the active ingredient and the water-insoluble polymer,
(b) melt-extruding at a temperature ranging from 60 to 220° C., and
(c) comminuting or grinding the extrudate to a granule.

13. A process for preparing a pharmaceutical composition according to claim 1 in the form of a solid comprising:
(a) solvating in an organic solvent or a solvent mixture the cationic, water-soluble (meth)acrylate copolymer, and the active ingredient and the water-insoluble polymer, and
(b) removing the organic solvent by evaporation or applying reduced pressure, to afford a solid with the named properties.

14. A process for producing a pharmaceutical form comprising:
(a) preparing a pharmaceutical composition in granulated or ground form by the process according to claim 12 and
(b) processing said pharmaceutical form to produce granules, pellets or powders, by mixing, compressing, powder layering, encapsulation, or a combination thereof.

15. A pharmaceutical form comprising a pharmaceutical composition according to claim 1.

16. (canceled)

17. The pharmaceutical composition according to claim 3, wherein the cationic, water-soluble (meth)acrylate copolymer comprising 30 to 80% by weight of C₁- to C₄-alkyl esters of methacrylic acid, and 70 to 20% by weight of an (meth)acrylate monomer having a tertiary amino group in the alkyl radical.

18. The pharmaceutical-composition according to claim 1, wherein the water-insoluble polymer is a copolymer comprising 20 to 40% by weight of ethyl acrylate and 60 to 80% by weight of methyl methacrylate and 0 to less than 5% by weight of methacrylic acid.

19. The process according to claim 14, wherein the pharmaceutical form is a tablet or a multiparticulate pharmaceutical form comprising tablets, minitablets capsules, sachets or reconstitutable powders.

20. The process according to claim 14, wherein said processing further comprises adding pharmaceutically customary excipients.

21. A process for producing a pharmaceutical form comprising:
(a) preparing a pharmaceutical composition in solid form by the process according to claim 13 and
(b) further comprising processing said pharmaceutical form to produce granules, pellets or powders, by mixing, compressing, powder layering, encapsulation, or a combination thereof.
